(19) United States
(12) Patent Application Publication
(10) Pub. No.: US 2003/0100019 A1
(43) Pub. Date: May 29, 2003

(54) Supported membrane, preparation and uses

(75) Inventors: Cathy Drexler, Bagnols/Ceze (FR); Patrick Berna, Palaiseau (FR)

Correspondence Address: MCKENNA LONG & ALDRIDGE LLP, 1900 K STREET NW, WASHINGTON, DC, 20006, US

(73) Assignee: WARNER-LAMBERT COMPANY, Morris Plains, NJ

(21) Appl. No.: 10/211,247

(22) Filed: Aug. 5, 2002

(30) Foreign Application Priority Data
Aug. 7, 2001 (FR) 01 10542

(51) Int. Cl. G01N 33/53; C12N 9/00; B28B 1/14

(52) U.S. Cl. 435/7.1; 530/412; 435/183; 264/299

(57) ABSTRACT

The present invention describes a new artificial supported membrane, as well as methods of preparation of such membrane or of reconstitution of cell membranes from their constituent proteins and lipids. Such membrane comprises a lipid bilayer attached to a support by phospholipid tethers (P) creating a space between the bilayer and the support, as well as one or more ligands (L) specific of a protein, covalently bound to the support and exposed in said space. The invention equally describes the uses of such membranes which allow the purification and/or reversible capture of membrane proteins or yet the screening of compounds that interact with some of these proteins. A membrane according to the invention may further serve to evaluate the toxicity or, conversely, the therapeutic effect of test compounds. The invention is particularly useful for analyzing protein-protein interactions within membranes and may thus be employed in the pharmaceutical industry for analysis of the toxic or beneficial profile of molecules that may be candidates for pharmaceutical development and/or enter into pharmaceutical compositions. In the field of biotechnology or in the field of medicine, such supported membranes may also be used in the manufacture of more efficient biomaterials.

The present invention describes a new artificial supported membrane, as well as methods of preparation of such membrane or of reconstitution of cell membranes from their constituent proteins and lipids. Such membrane comprises a lipid bilayer attached to a support by phospholipid tethers (P) creating a space between the bilayer and the support, as well as one or more ligands (L) specific of a protein, covalently bound to the support and exposed in said space. The invention equally describes the uses of such membranes which allow the purification and/or capture (possibly in a reversible manner) of membrane proteins or yet the screening of compounds that interact with some of these proteins. A membrane according to the invention may further serve to evaluate the toxicity or, conversely, the therapeutic effect of test compounds. The invention is particularly useful for analyzing protein-protein interactions within membranes and may thus be employed in the pharmaceutical industry for analysis of the toxic or beneficial profile of molecules that may be candidates for pharmaceutical development and/or enter into pharmaceutical compositions. In the field of biotechnology or in the field of medicine, such supported membranes may also be used in the manufacture of more efficient biomaterials.

Cell membranes are generally composed of a lipid bilayer whose components are held in place through intermolecular forces. Hydrophobic bonds in particular maintain the cohesion of the bilayer. A lipid is generally defined as a molecule comprising a hydrophobic hydrocarbon chain and a hydrophilic polar headgroup. Such membranes separate cellular compartments and are therefore exposed both to the inner compartment and the outer compartment of the cell.

Cell membranes are sites of signal transduction processes essential for cellular life. Specialized membrane receptors are specific binding sites of ligands thereby allowing transmission of extracellular signals through the membrane, their amplification and their integration. Supported lipid bilayers are therefore excellent models to study ligand-receptor interactions occurring at the surface of cell membranes.

The supported membranes described in the prior art comprise phospholipid bilayers immobilized on metallic surfaces, the proximal lipid layer located at some distance from said surface in such a way as to create an aqueous space within which membrane receptor molecules can be inserted (cf: WO 96/38726). Hydrophilic spacer molecules are thus used to maintain this aqueous space essential for the function of membrane-anchored proteins and for maintenance of membrane fluidity. The metallic surface may be coated with a SAM (Self-Assembled Monolayer), composed of molecules comprising long chains, preferably hydrocarbons and containing a number of carbon atoms that is generally greater than 10, which are sometimes interrupted by heteroatoms. Such chains carry a functional group allowing the membrane to be anchored to the support.

One of the main drawbacks of the known artificial membranes is that they are not suited for use in any type of purification, in so far as they do not allow retention or capture of the membrane proteins which one is seeking to purify for the purpose of enriching the artificial membrane in said membrane proteins.

Obtaining artificial membranes enriched in a molecular species (e.g., a membrane protein) has proved decisive for the study of said molecular species in its natural environment in a selective and specific manner. Selectivity and specificity are critical properties for the use of artificial membranes in screening tests of membrane proteins, a fortiori and in particular for screening tests of the high throughput type (HTS).

The present invention makes it possible, in particular thanks to the presence of tethers and molecules capable of retaining or capturing the molecular species of interest in its natural form or conformation, to obtain supported membranes enriched in said molecular species and in particular supported membranes comprising only said molecular species.

Furthermore, known membranes (WO 96/38726) present a constraint in terms of the size and arrangement of the spaces able to accept membrane proteins. The present invention makes it possible to modulate the architecture of the spaces able to accept membrane proteins by varying the arrangement and density of the tethers that support the membrane. This results in better control of membrane stability and fluidity over the entire supported membrane.

The present invention therefore proposes for the first time methods for the preparation of artificial membranes comprising a lipid bilayer anchored to a support by phospholipid (P) tethers that create an (aqueous) space between the bilayer and the support, and one or more ligands (L) specific of a protein (or a molecule of interest) covalently bound to the support and exposed in said space. Such ligands have the particular feature of considerably enhancing the specificity of binding to one or several specific types of proteins of interest.

The invention thus describes for supported membranes allowing specific binding of proteins of interest, in a functional configuration. This characteristic offers numerous advantages, including notably the use of the membranes for the purification of proteins from membrane preparations.

It is understood that <<purification of proteins from membrane preparations>> refers in particular to the enrichment in a molecular species of interest of a membrane preparation.

The present application equally describes the conditions of preparation of artificial supported membranes, conferring better stability and better fluidity of the membrane, for applications such as purification, screening, etc.

In this regard, one method of the invention comprises notably the following steps:

a) placing a functionalized surface in the presence of a mixture of specific ligands (L) and phospholipid tethers (P) able to bind to said functionalized surface, and

b) placing the surface obtained in a) in the presence of lipids so as to allow the assembly of a lipid bilayer supported by the phospholipid tethers (P).

The functionalized surface generally comprises molecules bearing functional groups allowing a covalent bond to be formed between the phospholipid tethers (P) and/or specific ligands (L), and molecules bearing non-functional groups.

The invention equally relates to the use of the supported membranes defined hereinabove, kits comprising them and methods of purification or screening of compounds implementing them.

To better understand the features of the invention and before describing the preferred methods by which to produce an artificial supported membrane, the nature of the components of such membrane shall first be described.

Nature of the Surfaces

The surfaces used are artificial surfaces. Usable materials include in particular surfaces composed of or based on materials such as gold, glass, diamond, silicon, silicon dioxide (SiO₂), silicon nitrite, tantalum pentoxide (Ta₂O₅), titanium dioxide (TiO₂), titanium nitrite, titanium carbide, platinum, tungsten, aluminium or indium-tin oxide, alone or in mixtures. Metallic supports, such as notably surfaces composed of or based on gold, are preferred.

An example of a usable support is a commercially available gold surface of the type Sia Kit Au (Biacore). This support can be analyzed by Surface Plasmon Resonance (SPR) and is compatible with the BIAcore apparatus.

The surfaces analyzed or used may adopt any useful functional forms and may, for example, be two-dimensional spaces, in the form of a square, rectangle or circle, preferably planar, or three-dimensional spaces, in the form of a cube, parallelepiped or sphere. Their areas range from 1 mm² to 100 cm². In a preferable manner, the area is comprised between 1 and 5 cm². Even more preferably, the area is comprised between 1 and 2.4 cm².

Description of the Functionalized Surface

The surface serving as the support is functionalized, i.e. preferably coated with a mixture of molecules bearing functional and non-functional groups. In a preferred manner, the surface is functionalized in the form of a SAM (Self-Assembled Monolayer). The surface may be functionalized with several types of functional groups able to interact, preferably in a covalent manner, with the (P) and/or (L) groups.

Some functional groups may further be activated so as to interact with the tether (P) and/or ligand (L).

Among the functional groups utilized, some can specifically bind the phospholipid tethers (P) while others specifically bind the specific ligands (L). This mode of implementation permits good control of the (P)/(L) ratios in the membrane. In this respect, the (P)/(L) ratio is preferably comprised between 0 and 10, more preferably between 0 and 4, and even more preferably between 0.01 and 1.

The type of functional group is chosen according to the coupling reaction used to anchor the tether (P) and the specific ligand (L). Such groups may correspond to a COOH, CHO, OH, NH₂ maleimide, or biotin group, for example. Following activation, these groups form a covalent bond of the amide (—NHCO—) or imide (—CHNH—) type if the (P) or (L) group bears an NH₂ function, a disulfide bridge (—S—S—) or thioether (—C—S—) if the (P) or (L) group bears an —SH bond or yet a stable biotin-streptavidin/avidin complex when the (P) or (L) group is functionalized with streptavidin or avidin.

One of the preferred functional groups according to the invention is the COOH group. The latter may be activated and thereby give rise to formation of an ester by reaction in aqueous medium with N-hydroxysuccinimide (NHS) and in the presence of dicyclohexylcarbodiimide (DCC).

In a first specific embodiment of the invention, the functionalized surface comprises functional groups X that can possibly be activated, capable of anchoring the specific ligands (L) and phospholipid tethers (P), and non-functional groups Z.

The term non-functional group refers to any molecule incapable of reacting (i.e., covalently and specifically binding) with a (P) or (L) molecule. It may be any nonreactive molecule or groups such as noted hereinabove, but which are not previously activated.

In a second specific embodiment, the surface is coated with a mixture of molecules comprising molecules bearing functional groups X and Y, that can possibly be activated, capable of covalently and specifically binding the phospholipid tethers (P) and/or the specific ligands (L).

In another specific embodiment of the invention, the functionalized surface comprises a mixture of molecules bearing functional groups X and Y, the groups X and Y selectively binding the specific ligands (L) and the tethers (P), respectively.

In the preparation of the membranes disclosed in the invention, the molar ratio of functional groups X and Y to non-functional groups Z is generally comprised between 0.05 and 20, preferably between 0.1 and 5. In an especially advantageous manner, this ratio is less than 1 and, more preferably, comprised between 0.1 and 0.3. The studies carried out indicate that these preferred ratios confer sufficient stability all while allowing the insertion, in the membranes, of important proteins or protein complexes.

The molecules used to coat the surface may be thiol derivatives represented by the formula HS—(CH₂)ₙ—X, HS—(CH₂)ₘ—Y or HS—(CH₂)ₚ-Z.

The advantage of using a mixture of at least two thiol derivatives is to be able to control the density of functional groups present at the surface and thereby to favor the capture of a membrane protein which is more or less large in size, the stability of the membrane and its fluidity.

The thiol derivatives may differ by the nature of their chain and, as indicated hereinabove, by the type of functional group. Thus, the length of the carbon chain varies as a function of the numbers n, m and p. Generally, n, m and p are comprised between 2 and 15, more preferably between 8 and 15 and, in a preferred variant, they are equal to 10 or 11. The length of the chain may be adjusted by those skilled in the art.

It is desirable that all molecules bearing functional groups are more or less equal in length, so as to obtain a homogeneous surface. For this reason, the numbers n, m and p should preferably be essentially the same or similar, particularly not differing by more than ±2. Especially preferred is the use of the molecules defined hereinabove, wherein n, m and p are the same whole numbers.

The nucleophilic groups that bind to the support surface may be thiols as described hereinabove but they may also be primary, secondary or tertiary amines bound directly or by means of a spacer whose length is necessarily shorter than that of the spacer of the phospholipid tether (the latter shall be described in more detail below). In addition to amines, they may also be hydrazide or carboxylate groups. In these latter cases, the lipids placed in contact with this surface must bear polar headgroups modified by electrophilic groups such as phosphatidyl esters, halide esters or acids or even carbonyl, epoxide or vinyl groups. The advantage of thiol, pyridyl disulfides, maleimides or haloacetate functions is that they give rise to selective reactions. Such functions are therefore preferred in the scope of the invention.

Description of the Phospholipid Tethers (P)

The tethers disclosed in the invention preferably comprise three parts, namely, an anchorage point to the functionalized surface, a spacer and a phospholipid.

In fact the tethers bind to the SAM through the functional groups (activated to this effect), described hereinabove. As noted, the anchorage point comprises a reactive group that can react with a functional group on the functionalized surface. Such functional group may be a COOH, CHO, OH, NH₂, maleimide or biotin group, for example.

One object of the present invention therefore relates to particularly advantageous methods in which the phospholipid tether (P) comprises a point of anchorage to a functionalized surface, a spacer—situated between the anchorage point and the polar headgroup of the phospholipid—and a phospholipid. The spacer usually corresponds to a hydrophilic chain comprising from 2 to 200 carbon atoms, the length of the spacer being less than 500 Å, preferably comprised between 3 and 500 Å. Spacers having a length of approximately 3.5-5 Å, 11-14 Å or 170-220 Å, may be cited as examples.

So as to obtain a planar surface, the tethers must have more or less the same length.

The phospholipid tether may be used in several forms:

it may be an aqueous solution of the phospholipid in the case where the latter has sufficient water solubility

it may also be a micelle solution (lipid/detergent mixture) or liposomes when the phospholipid is insoluble in aqueous medium.

The phospholipid head of the tether serves as the point of attachment of the bilayer. It may correspond to different phospholipids, such as the phospholipids present in the membranes of mammalian cells or of plant or bacterial cells, etc. For example, it may be phosphatidylcholine (PC), phosphatidylserine (PS), phosphatidylethanolamine (PE), phosphatitylinositol (PI), phosphatidylglycerol (PG), etc.

Different commercially available phospholipid tethers may be utilized in the invention, which differ from each other in terms of the length (I) of the spacer and the structure of the phospholipid fatty acid chain. Among the commercially available phospholipid tethers that may be used in the scope of the invention, specific examples include PE, comprising a spacer whose length varies between 3 and 5 Å, DOPE-C comprising a spacer whose length varies between 11 and 14 Å, PEG-DPPE comprising a spacer whose length varies between 176 and 214 Å (FIG. 2).

Description of the Specific Ligands (L)

The specific ligand (L) comprises a group with specific affinity for a specific molecule and an anchorage point to the functionalized surface. In a particular embodiment, the ligand further comprises a spacer. The spacer of the ligand may correspond to a hydrophilic chain comprising from 2 to 200 atoms and is preferably chosen so that the length of the specific ligand is less than that of the phospholipid tether.

The affinity group of the ligand (L) may be a natural, synthetic or chemical product displaying affinity for a molecule of interest, such as notably a protein and more specifically a membrane protein, a membrane receptor (able to bind to an inhibitor or effector ligand), an immunoglobulin, a lectin, an antigen, a hapten or a substrate, etc., such molecules being able to specifically bind to a respective ligand. In a preferred embodiment of the invention, the specific ligand L corresponds to a ligand with specific affinity for a protein constituent. Advantageously it is a specific antibody or antibody fragment (e.g., Fab, Fab′2, CRD, ScFv, etc.) of a protein or protein complex, particularly of at least one extramembrane part of a protein or protein complex.

The ligand may also be a protein known to have specific affinity for a class of molecules such as a lectin, streptavidin, A protein, G protein or more broadly for a protein that is part of a multiprotein complex. According to another preferred variant, L corresponds to a chemical molecule of low molecular mass displaying affinity for the protein of interest to be bound. Such chemical molecule may be an inhibitor, activator, agonist, antagonist, effector or cofactor of such protein.

A membrane protein present in a natural vesicle or in membrane fragments may thus be bound to the support, according to an especially advantageous method, by reaction with the specific ligand. The bound membrane protein may be purified by introducing a flow of phospholipid at the surface of the membrane so that unbound proteins are eluted or swept away.

Moreover, in the construction of a membrane according to the invention, it is possible to use several ligands (L) having affinity for different molecules of interest. These may be two membrane receptors, a receptor and a co-receptor, a receptor and a G protein, several receptor subunits, etc.

The specific ligands (L) are anchored to the SAM through the functional groups described hereinabove. As noted, the point of anchorage comprises a reactive group that can react with a functional group on the functionalized surface. When the functional group is a COOH, CHO, OH, NH₂, maleimide or biotin group, the reactive group may be an SH, NH₂ group, avidin or streptavidin, for example.

The spacer of the specific ligand (L) may equally correspond to a hydrophilic chain. The spacer length is chosen so that the length of the specific ligand (L) is less than that of the tether (P). Preferably, the spacer comprises from 2 to 50 atoms. The spacer length is advantageously less than 500 Å and is preferably between 3 and 500 Å and even more preferably between 3 and 400 Å. Spacers with lengths comprised between 3 and 5 Å, between 10 and 20 Å or between 150 and 220 Å may be given as examples. The length of the spacer of the ligand (L) is preferably less than the length of the spacer of the tether (P).

Principle of the Coupling Reaction

Coupling may be accomplished by means of methods known in themselves, generally comprising the following steps:

A first, optional step to activate the functionalized surface,

A step to couple the functional groups to tethers (P) and to specific ligands (L), and

An optional step to inactivate unreacted functional groups.

The X and/or Y groups on the functionalized surface are possibly activated before the fixation of the tethers and ligands. Activation of the functional group is not always necessary and depends on the nature thereof. Taking the COOH group as an example, activation consists in reacting this functional group in aqueous medium and in the presence of dicyclohexylcarbodiimide (DCC) with N-hydroxysuccinimide (NHS), so as to form an ester. X is then able to react with L or P indifferently.

The Y group may then correspond to a non-functional hydroxyl group (OH) which does not take part in the coupling reaction.

Another possibility disclosed in the invention consists in activating both X and Y groups and introducing in the mixture a molecule bearing a non-functional group Z, incapable of binding selectively and covalently with an (L) or (P) molecule. The interest of introducing a molecule bearing a non-functional group is to preserve free spaces between the phospholipid tethers, wherein proteins of interest will be able to insert themselves by binding to the specific ligands (L).

The coupling reaction is carried out in one or two steps

P and L may be co-coupled, i.e. simultaneously coupled, or P and L may be coupled in two successive steps via the functional groups of each.

The number of L or P molecules anchored to the surface of the support depends either on the amounts of (P) and (L) used, when coupling occurs via specific functional groups, or on the molar ratio of (L) and (P) present in the mixture, or on the concentration of the functional groups that can react specifically with (L) and (P).

The situation where X corresponds to the functional group and Z to the non-functional group may be represented by the following formula

aX+bZ→aX*+bZ→(a−c)P+cL+bZ

wherein a=number of X groups, X*=activated X group, b=number of Z groups, c=number of ligands, (a−c)=number of tethers (P) and a+b=100.

The case where the functional groups X are activated then successively placed in the presence of the tether (P) and the specific ligand (L) allows better control of the density of the tethers anchored to the surface of the SAM and may be represented by the following formula:

aX+bZ→aX*+bZ→(a−c)P+cX*+bZ→(a−c)P+cL+bZ

The situation where the functional groups X are activated and react with the tether (P) may be represented by the following formula, unreacted X groups being inactivated by means of a molecule capable of binding with a specific ligand (L).

aX+bZ→aX*+bZ→(a−x)P+xX*+bZ

(a−x)P+xX*+bZ→(a-x)P+xX′+bZ→(a−x)P+xL+bZ

The case where the X and Y groups after activation react with the tether (P) and the ligand (L), respectively, may be represented by the following formula:

aX+bY→aX* +bY→aP+bY→aP+bY*→aP+bL

Activated groups which did not react undergo an inactivation reaction. In the case of carboxylate groups, ethanolamine (Biacore) may be used as an inactivation mean.

Source and Components of the Lipid Bilayer

The lipid bilayer of the membranes disclosed in the invention may comprise lipids of diverse nature and origin. Such lipids may come from cell extracts, membrane fragments, lipid monolayers or bilayers, liposomes or lipid vesicles and/or lipids, phospholipids, lipopeptides, biotinylated lipids and their mixtures. The bilayer is formed by assembly of the lipids with the lipids of the tether (P). The amount of lipids used, regardless of their form, is adapted by those skilled in the art.

The planar and continuous membranes prepared according to the invention may be produced in an apparatus such as the Biacore so as to maintain a constant flow of solvent and/or solute and/or lipid vesicles at the membrane surface. This principle lowers the risks of contamination and of membrane denaturation or destruction.

In a preferred manner, the construction of the membrane is envisioned according to two methods which consist in a dilution of micelles or a fusion of vesicles.

Methods Yielding a Supported Membrane

The present invention describes several methods by which to produce a supported membrane. Generally speaking, they comprise the following steps:

a) placing a functionalized surface in the presence of a mixture of specific ligands (L) and phospholipid tethers (P), able to bind to said functionalized surface, and

b) placing the surface obtained in a) in the presence of lipids so as to allow the assembly of a lipid bilayer supported by the phospholipid tethers (P).

This method distinguishes among the functional groups the X groups that can possibly be activated, capable of coupling with the specific ligands (L) and/or the phospholipid tethers (P), as well as the non-functional groups Z.

According to another variant of the invention, the functional groups X and Y may both be activated so as to specifically bind (anchor) the specific ligands (L) and/or phospholipid tethers (P).

In a particular method, the surface comprises a mixture of molecules bearing functional groups X and Y, the X and Y groups selectively binding the specific ligands (L) and tethers (P), respectively.

As explained hereinabove, the molecules bearing functional groups may be thiol derivatives represented by the formula HS—(CH₂)n-X or HS—(CH₂)m-Y, n and m being comprised between 2 and 15.

Another method according to the invention yields a supported membrane. It comprises the following steps:

a) coating a surface with molecules represented by the formula HS—(CH₂)n-X and HS—(CH₂)m-Z, X being a previously activated functional group capable of coupling with a specific ligand (L) or a phospholipid tether (P), Z being a non-functional group, n and m being an integer comprised between 2 and 15,

b) placing the surface obtained in a) in the presence of a mixture containing the specific ligands (L) and the phospholipid tethers (P), then

c) placing the surface obtained in b) in the presence of lipids so as to allow the assembly of a lipid bilayer supported by the phospholipid tethers (P).

According to a further method of the invention, it is possible to produce a supported membrane comprising the following steps:

a) coating a surface with molecules bearing functional groups represented by the formula HS—(CH₂)n-X and HS—(CH₂)m-Z, X being a previously activated functional group capable of coupling with a specific ligand (L) or a phospholipid tether (P), Z being a non-functional group, n and m being an integer comprised between 2 and 15,

b) placing the surface obtained in a) in the presence of phospholipid tethers (P), then

c) placing the surface obtained in b) in the presence of specific ligands (L), and

d) placing the surface obtained in c) in the presence of lipids so as to allow the assembly of a lipid bilayer supported by the phospholipid tethers (P).

Another method comprises the following steps:

a) coating a surface with molecules bearing non-functional groups and molecules bearing functional groups represented by the formula HS—(CH₂)n-X, HS—(CH₂)m-Y and HS—(CH₂)n-Z, X and Y being previously activated functional groups capable of coupling with a specific ligand (L) and a phospholipid tether (P), respectively, Z being a non-functional group, n and m being an integer comprised between 2 and 15,

b) placing the surface obtained in a) in the presence of a mixture containing specific ligands (L) and phospholipid tethers (P), then

c) placing the surface obtained in b) in the presence of lipids so as to allow the assembly of a lipid bilayer supported by the phospholipid tethers (P).

The methods described hereinabove may comprise an additional step after step a) to inactivate X and Y groups that did not react with the ligands (L) or tethers (P). Such inactivation may be performed by any known method, according to the nature of the X or Y group, and for example by molecules capable of binding to the X or Y groups.

Moreover, in a particular embodiment, the method comprises an additional step to insert a molecule of interest into the membrane. This step may be carried out at the same time as the assembly of the bilayer, or thereafter. When this step is carried out after assembly of the bilayer, it is preferably executed by fusion of a natural vesicle containing the protein of interest, with the previously assembled bilayer.

The invention encompasses any artificial membrane obtained according to any of the methods disclosed hereinabove.

Applications of Supported Membranes

The membranes according to the invention therefore represent an ideal model to study exchanges across cells, without having to resort to animal experimentation but remaining close to the in vivo situation. In particular, they enable analysis of protein-protein interactions within the membrane, by using purified proteins, under physiological conditions.

SPR analysis is an optical method which, like ellipsometry and evanescent wave spectrosocy (EWS), Brewster angle refractometry, critical angle refractometry, Frustrated Total Reflexion (FTR), evanescent wave ellipsometry, Scattered Total Internal Reflexion (STIR), etc., allows measurement of surface concentrations or refractive index. These parameters indeed vary in relation to membrane composition and thus permit the study thereof.

The supported membranes of the invention may advantageously be used for the reversible capture of membrane proteins, complexed or not, by means of their specific ligands.

In this context, the specific features of the artificial membranes according to the invention have made it possible to develop a method of purification of proteins comprising (a) the placing in contact of a supported membrane as described hereinabove and/or obtained by any of the methods of the invention, with a membrane preparation comprising a protein to be purified, under conditions permitting insertion of said protein to be purified into said membrane, and (b) applying a flow of lipids. This method can of course be used to purify any other molecule contained in a membrane extract. Such method is based in particular on the use of a membrane according to the invention comprising a specific ligand (L) of said molecule.

The transmembrane proteins are immobilized directly or indirectly in a lipid bilayer by means of the specific molecule (antibody, interaction protein, substrates, inhibitors, etc.) which constitutes the specific ligand (L). Proteins that did not bind may be eluted by a flow of phospholipids in the form of micelles or liposomes. This method is an effective means to purify transmembrane proteins under gentle, non-denaturing conditions.

The transmembrane proteins thus purified may then be used to develop screening tests for active molecules based on membrane receptors. The present invention therefore comprises the use of a supported membrane obtained by any one of the methods of the invention, to screen compounds interacting with native membrane proteins inserted in said supported membrane.

The supported membranes according to the invention which are enriched in one or more specific types of protein may be used notably in the manufacture of biosensors capable of converting a biological activity into a quantifiable signal. The biomaterials used in this manner are intended to be placed in contact with body fluids and tissues and must therefore comply with compatibility criteria. In fact, proteins and cells are known to adsorb to the surface of artificial materials when the latter are placed in contact with blood or other biological fluids, leading to undesirable effects. The existence of a membrane fitting into this particular physiological context would make it possible to avoid or to minimize such undesirable effects.

In a specific mode of implementation of the methods of study of reconstituted tissues according to the invention, the latter may be treated with one or more test molecules so as to characterize the membrane reactions towards such test molecule(s).

The test molecule may be quite varied in nature. For instance, it may be an individual molecule or a mixture of substances. The compound may be chemical or biological in nature. In particular it may be a peptide, polypeptide, nucleic acid, lipid, carbohydrate, chemical molecule, plant extracts, combinatorial libraries, etc.

To implement the method of the invention, the test compound may be applied at different concentrations, chosen by the user.

The present invention therefore provides for the use of a reconstituted membrane such as defined hereinabove, for the evaluation of the biological and/or toxic properties of a test molecule, particularly a molecule intended for therapeutic or cosmetic use, etc.

This method of study of reconstituted membranes may further allow analysis of the transcripts of different cell types as well as analysis of electrical activity, study of ion channels involved in exchanges and/or that of receptors, by electrophysiological methods or patch-clamp techniques.

The feasibility, execution and other advantages of the invention are illustrated in greater detail in the following examples, which are given for purposes of illustration and not by way of limitation.

### LEGENDS OF FIGURES

FIG. 1: Method of binding of thiol derivatives (incubation in ethanol solution at 1 mM at 4° C. for 16 hours, wash with ethanol solution, sonicate two times for 1 minute, binding to the gold surface of Biacore support).

FIG. 2: Structure of phospholipid tethers.

FIG. 3: Example of a phospholipid tether (POPC). A micelle solution containing 2 mg of POPC in 1 ml of a 50 mM octyl-β-D-glucoside (OG) solution and in HBS-N buffered medium (0.01 M HEPES, 0.15 M NaCl, pH 7.4) is placed in the presence of the functionalized surface.

FIG. 4: Results obtained for each surface used placed in the presence of a reagent.

(I) is signal intensity in RU before injection of a reagent and (F) after injection; the variation (A) of the signal corresponds to the difference (F−I). Intensities are given for injection of BSA before membrane formation (injection 2), POPC micelles (injection 4), NaOH (injection 5 and 6), BSA after formation of the POPC membrane (injection 7) and BSA after regeneration of the surface (injection 9).

FIG. 5:

a) Formation of POPC membrane on Sia-0.

b) Formation of POPC membrane on Sia-11.

c) Formation of POPC membrane on Sia-20.

d) Formation of POPC membrane on Sia-33.

e) Formation of POPC membrane on Sia-70.

f) Formation of POPC membrane on Sia-100.

FIG. 6: Effect of pH on the anchoring step.

FIG. 7: Effect of pH on the anchoring step.

FIG. 8: Anchoring of DOPE-C lipid on different surfaces.

FIG. 9: Anchoring of PEG-PE lipid on different surfaces.

FIG. 10: RU monitoring of the steps involved in formation of a supported membrane.

### MATERIALS AND METHODS

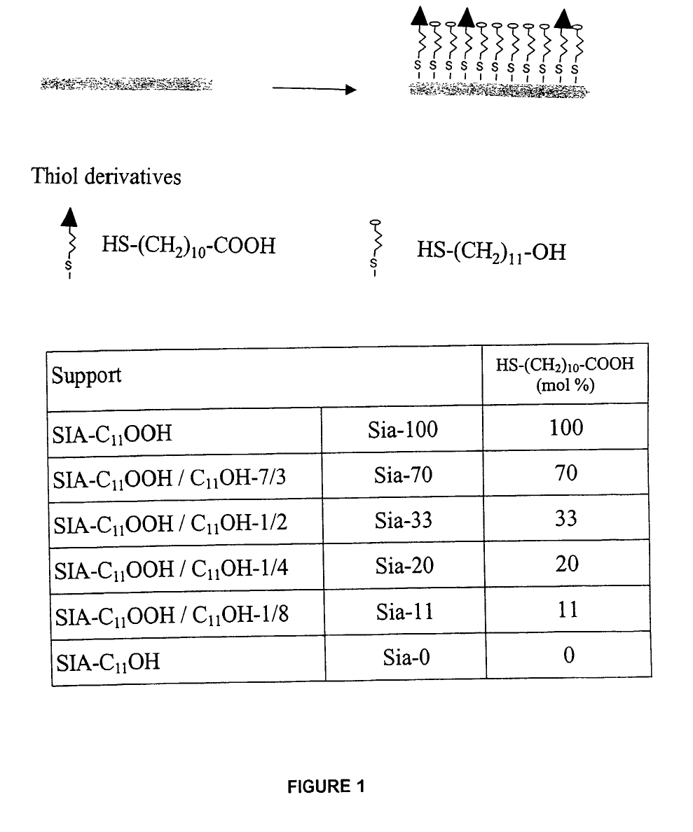

In this example a commercially available gold surface Sia Kit Au (Biacore) was used. Six different supports (Sia-100, -70, -33, -20, -11, -01) were prepared by spontaneous reaction of a 1 mM solution of ethanol containing the mixture of two commercially available thiol derivatives used at different molar ratios with respect to the gold surface (FIG. 1).

The thiol derivatives concerned are as follows:

11-mercaptoundecanoic acid (HS—(CH₂)₁₀—COOH)

11-mercapto-1-undecanol (HS—(CH₂)₁₁—OH)

Rationale for choosing the above thiol derivatives: the —COOH functional groups serve to anchor the tether. By varying the concentration of —COOH groups exposed at the SAM surface, the density of the tethers can be controlled.

The methods of covalent binding of molecules to the functionalized support by means of a SAM are similar to those generally employed for preparation of an affinity chromatography matrix. The coupling reaction may be carried out inside or outside the Biacore apparatus.

In our case, the —COOH group was reacted with the tether inside the apparatus it allows the efficiency of the coupling reaction to be monitored by RU signal intensity (Biacore unit).

The tether is anchored through a covalent amide bond

a) The —COOH group is activated to an ester by reaction in aqueous medium with N-hydroxysuccinimide (NHS) and in the presence of dicyclohexylcarbodiimide (DCC).

b) The phospholipid tether reacting with the activated ester may be used in several forms:

an aqueous solution of the phospholipid when its water solubility is sufficient, and for phospholipids insoluble in aqueous media:

in the form of a micelle solution (lipid/detergent mixture),

in the form of liposomes.

The preparation of micelles requires the use of detergent. We chose octyl glucoside (OG), a nonionic detergent easily removed by washing with water (high cmc), and whose structure contains —OH groups unreactive towards the activated ester.

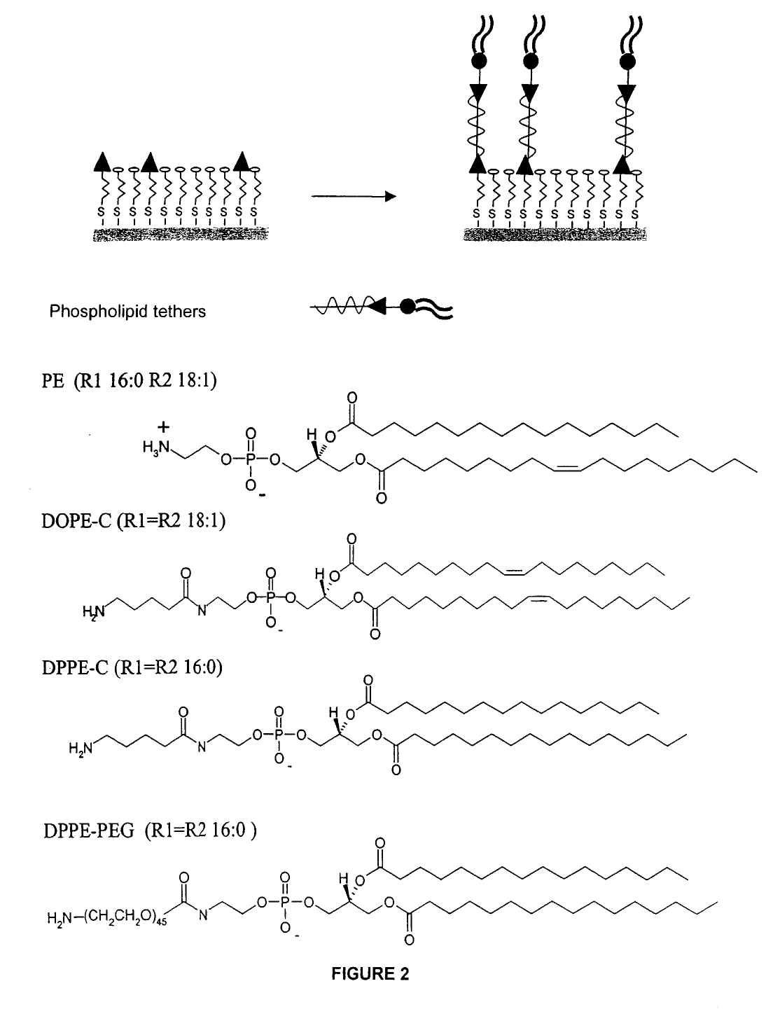

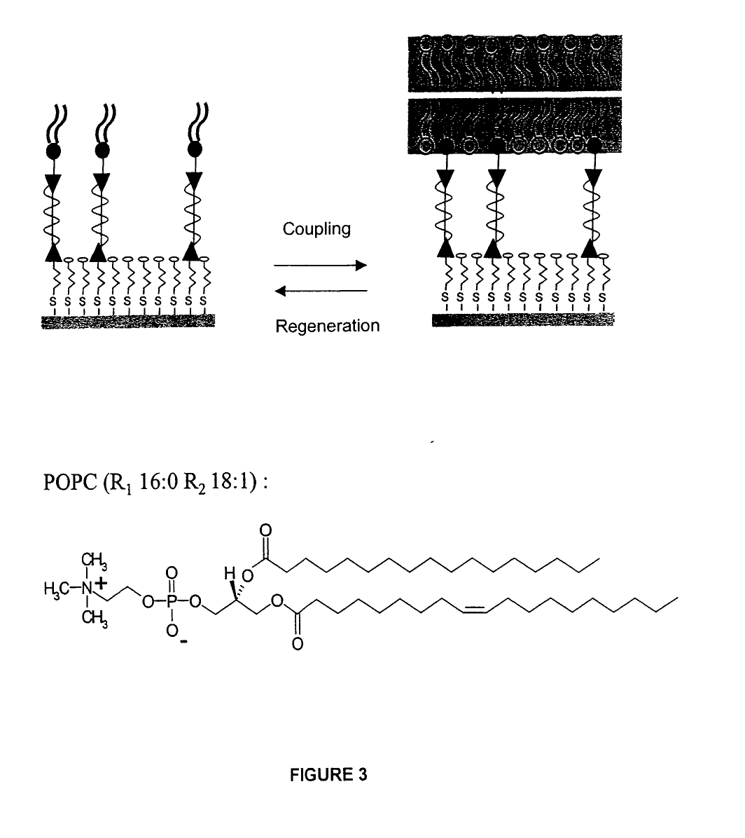

The structures of the different phospholipids are depicted in FIG. 2. These phospholipids are commercially available. They differ in terms of the presence or not of a spacer and the nature of the aliphatic chain (length, degree of unsaturation). The junction between the spacer and the polar headgroup of the phospholipid is of the amide type. The spacer may be elongated by successive addition of fragments via an amine bond (FIG. 3).

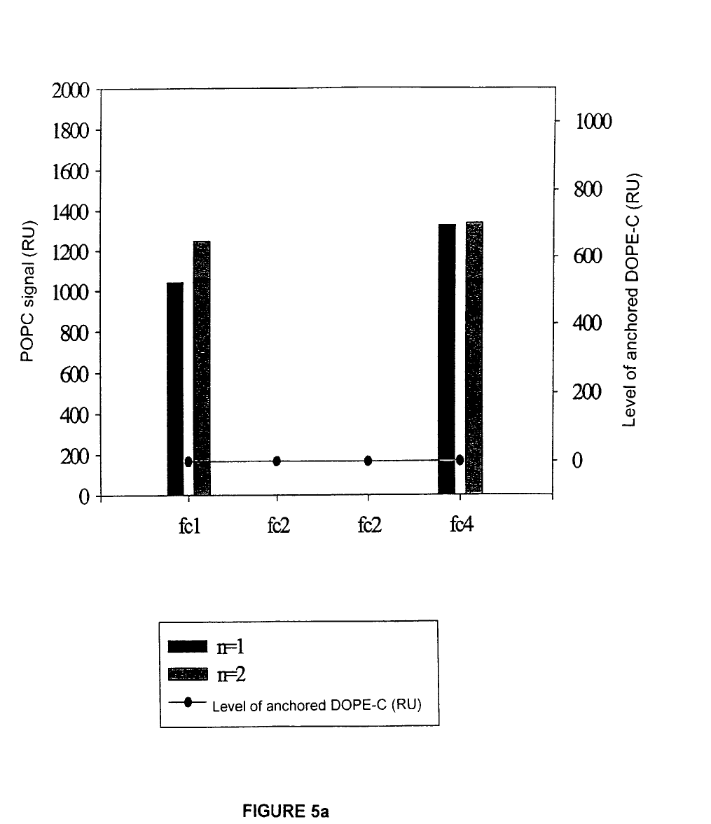

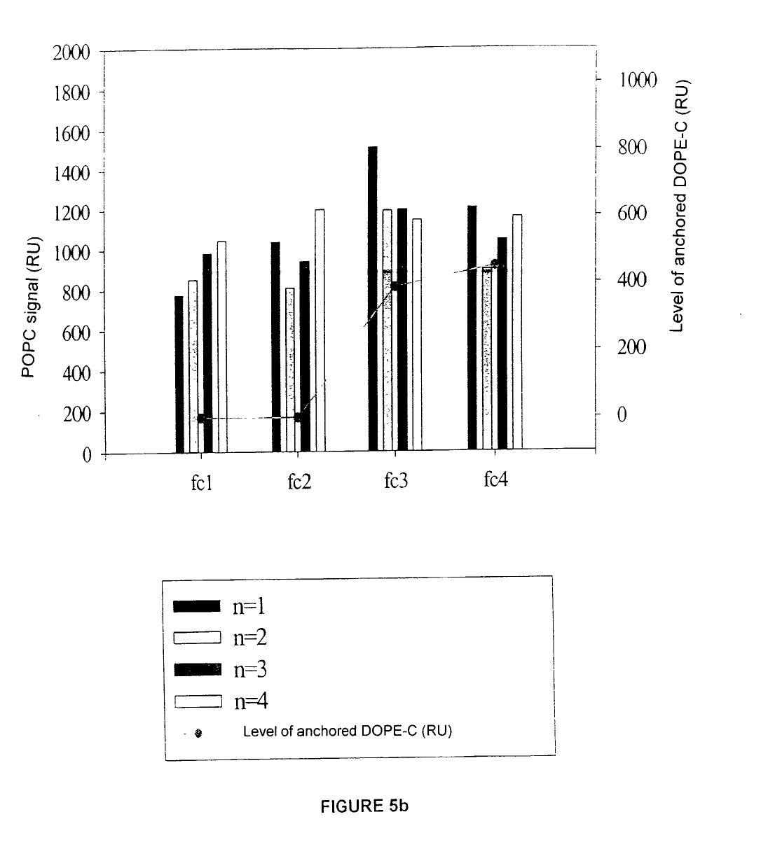

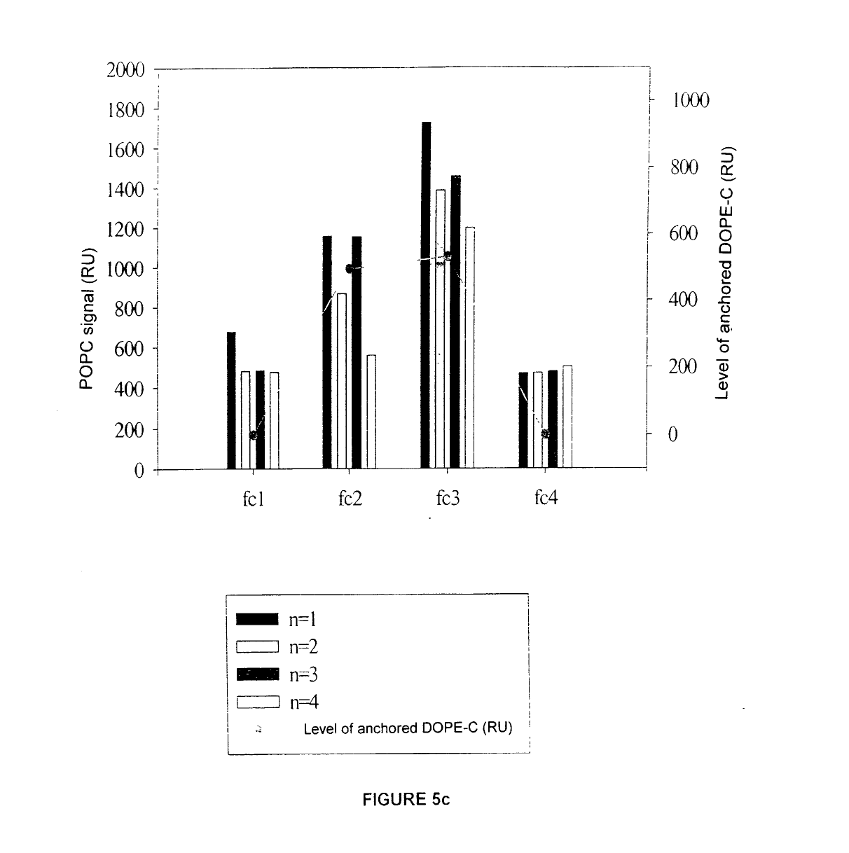

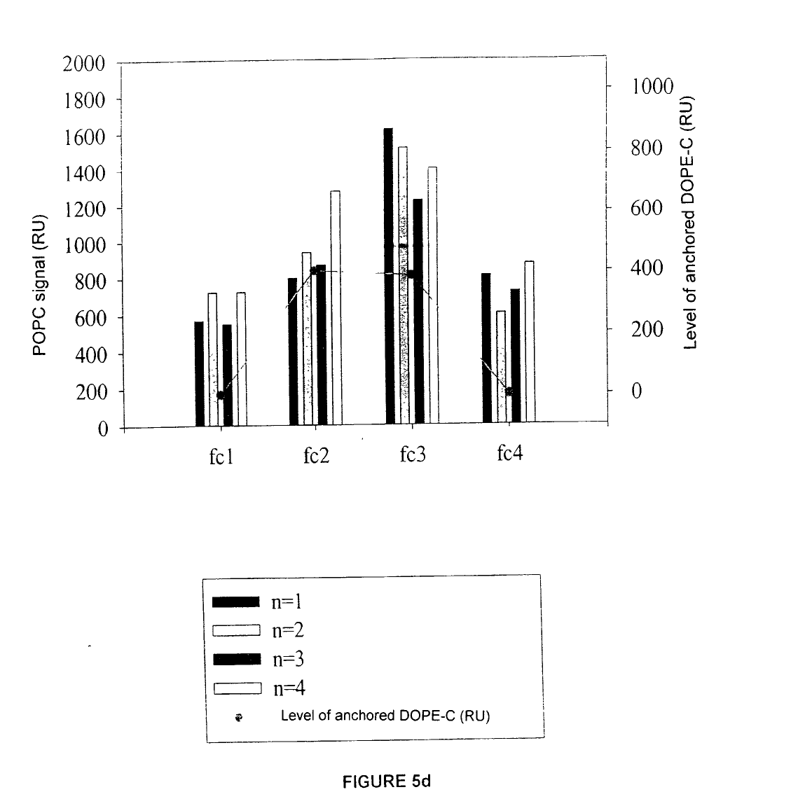

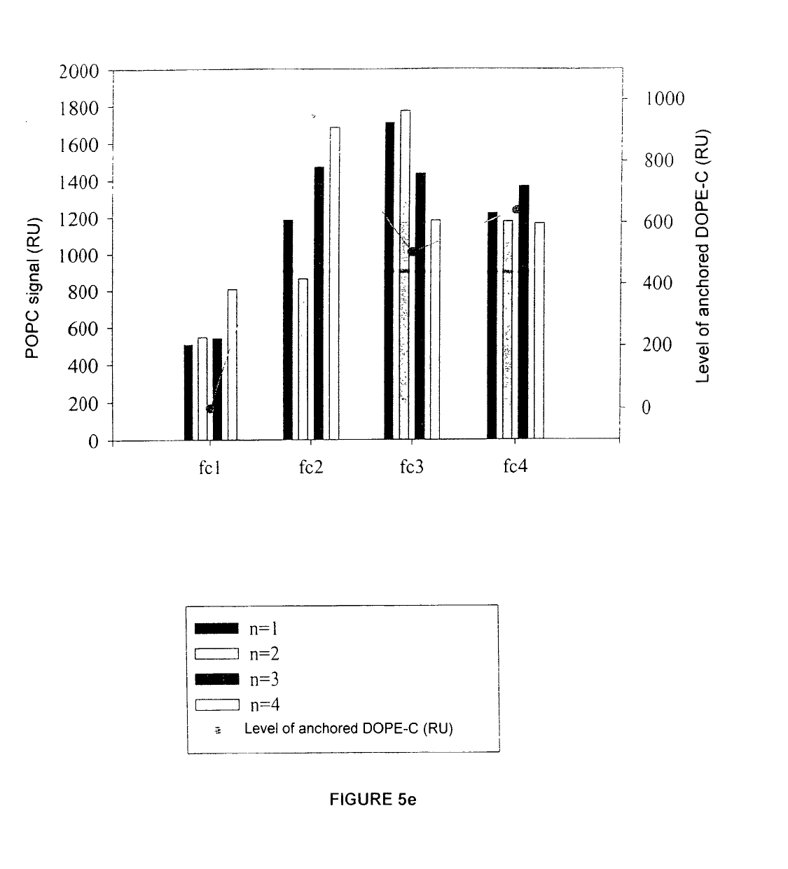

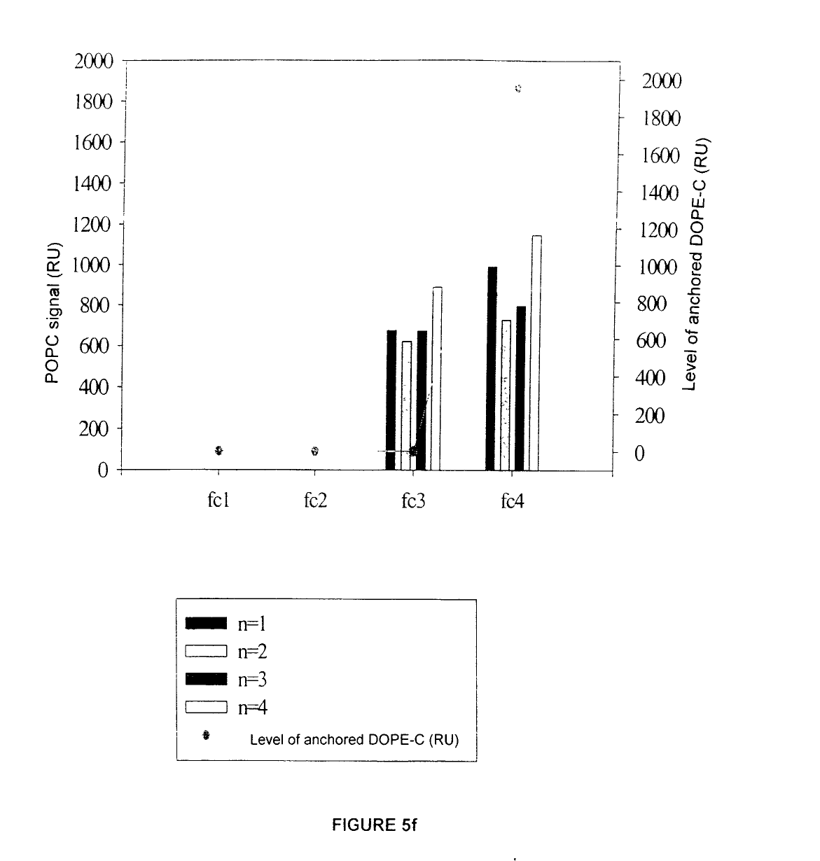

c) Carboxylate groups that did not react with the phospholipid are inactivated in the presence of ethanolamine. Formation of membrane on different supports using the hereinabove lipids is depicted in FIG. 5.

### EXAMPLE 1

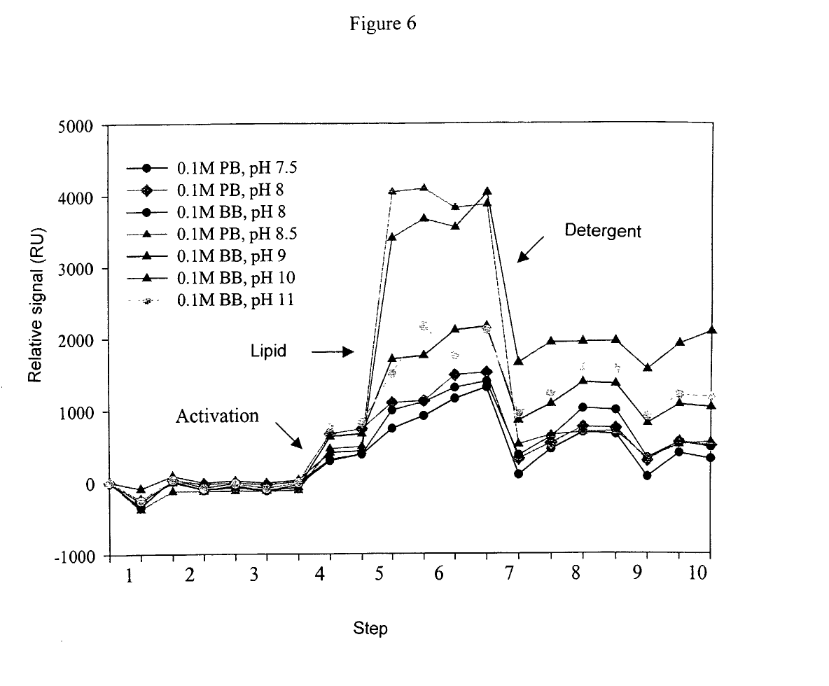

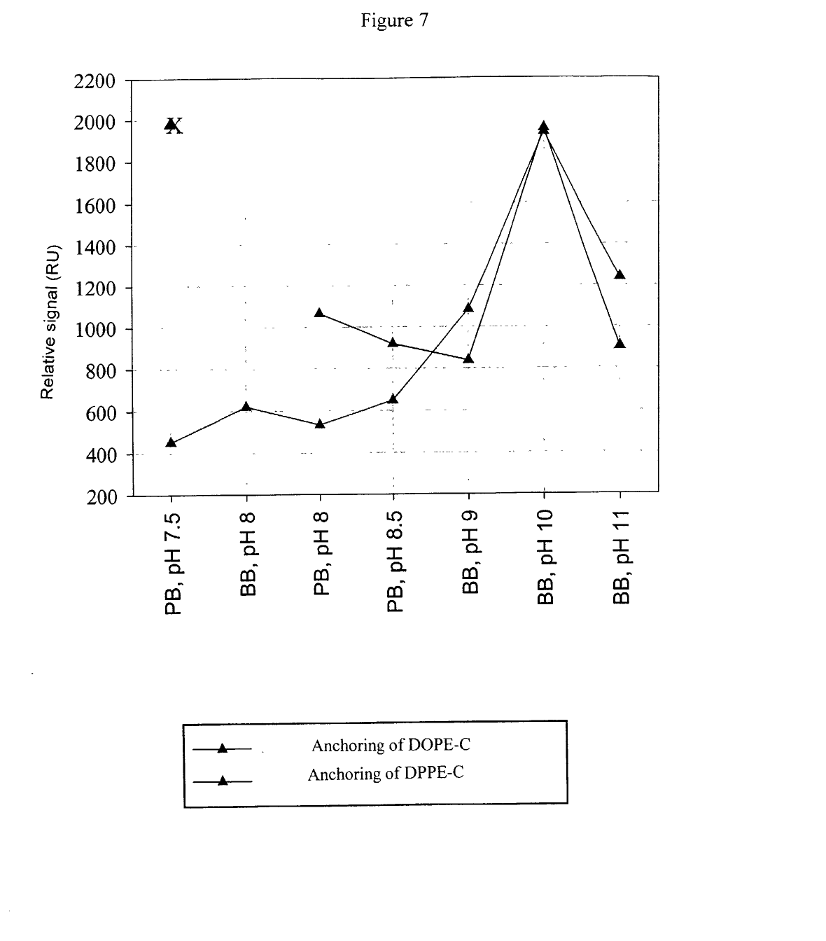

### (FIGS. 6 and 7)

Different pH conditions were tested so as to optimize the phospholipid coupling step (FIG. 6).

The procedure described below was implemented in an entirely automated manner:

| | | |
|---|---|---|
| 1. | 50 mM OG detergent in buffer X | (100 μl; 10 min) |
| 2. | 50 mM NaOH | (10 μl; 1 min) |
| 3. | 50 mM NaOH | (10 μl; 1 min) |
| 4. | EDC/NHS | (100 μl; 10 min) |
| 5. | Lipids (2 mg/ml of 50 mM OG in buffer X) | (100 μl; 20 min) |
| 6. | Lipids (2 mg/ml of 50 mM OG in buffer X) | (100 μl; 20 min) |
| 7. | 50 mM OG detergent in buffer X | (10 μl; 1 min) |
| 8. | Ethanolamine | (60 μl; 6 min) |
| 9. | 50 mM OG detergent in buffer X | (10 μl; 10 min) |
| 10. | 50 mM NaGH | (10 μl; 1 min) |

The surface is conditioned (injections 1-3), activated (4), the lipid is anchored (injection 5 and 6), unreacted lipid is removed by injection of detergent (7), then the carboxylate groups are inactivated (8). Another injection of OG removes adsorbed ethanolamine.

The pH was tested over a range from 7.5 to 11 using 0.1 M phosphate buffer (PB) or borate-NaOH buffer (BB). The phospholipid DOPE-C is injected as a micellar solution (2 mg/ml of 50 mM OG) in the different buffers on the Sia-100 surface (100%-COOH groups). Plot 1 shows the RU signal intensity before and after each injection of reagent. The intensities are relative because signals 1 and 4 obtained before injection are taken arbitrarily as reference and are therefore equal to 0. The level of coupling gradually increases to reach a maximum at pH 10. Similar results were obtained with the lipid DPPE-C (FIG. 7).

### EXAMPLE 2

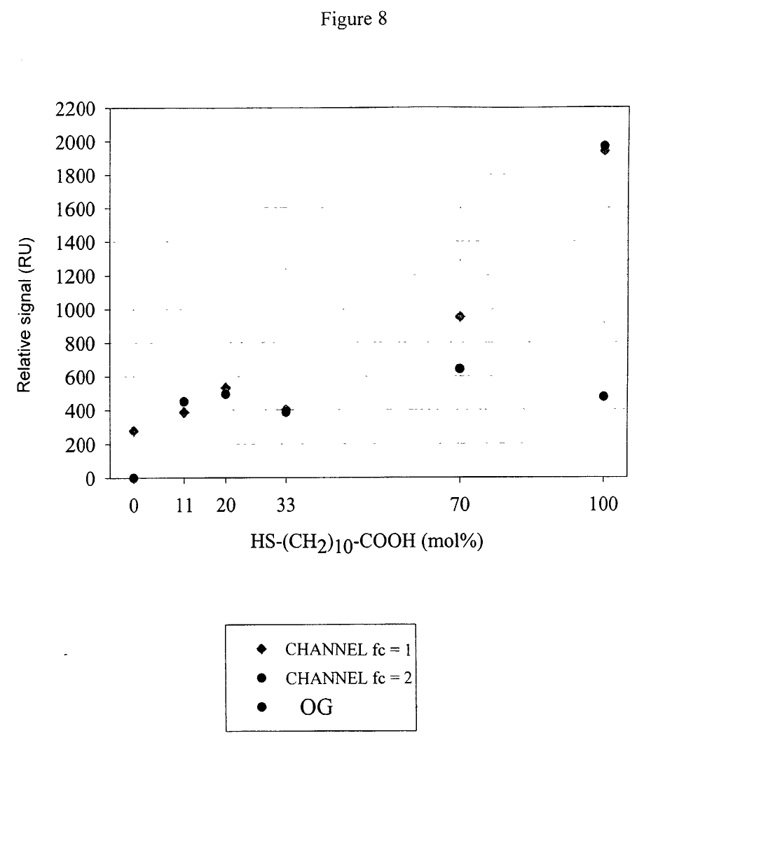

In a second example, the lipid DOPE is anchored at pH 10 on six surfaces having different concentrations of —COOH groups, according to the method described in example 1. The results are given in FIG. 8.

FIG. 8 gives the RU signal intensity observed before the inactivation step (injection 8) for all the surfaces Sia-0, -11, -20, -33, -70, -100%. The coupling reaction is carried out on two different channels (fc=1, fc=2) for a given surface.

For fc=1 and fc=2 the amount of DOPE-C anchored to the surface is similar for all the surfaces except Sia-70%. Coupling is greatest for Sia-100% then decreases by half for Sia-70% to reach a minimum for Sia-33, -20, -11 and Sia-0%.

Control Experiments:

In the presence of DOPE-C, the signal observed for Sia-0% which has no COOH groups that can be activated may be due to intercalation of the phospholipid. In fact, no signal is observed when only OG detergent is injected. In the case of Sia-100%, the signal may be due to baseline stray during the experiment. In the case of partial binding of the detergent, the method consisting in injection of a liposome solution containing the lipid to be bound overcomes the problem.

### EXAMPLE 3

The same experiment as in example 2 was conducted with PEG-PE phospholipid (MW 2739.37 g/mol) according to the following procedure:

| | |
|---|---|
| 50 mM NaOH | 10 μl (1 min) |
| 50 mM NaOH | 10 μl (1 min) |
| EDC/NHS | 100 μl (10 min) |
| PEG-PE (5 mg/ml in BB buffer, pH 10) | 50 μl (10 min) × 3 |
| Ethanolamine | 60 μl (6 min) |
| 50 mM NaOH | 10 μl (1 min) |
| 50 mM NaOH | 10 μl (1 min) |

It was not necessary to use OG detergent because the PEG-PE phospholipid is soluble in water. Non-covalently bound PEG-PE was chased with HBS-N buffer.

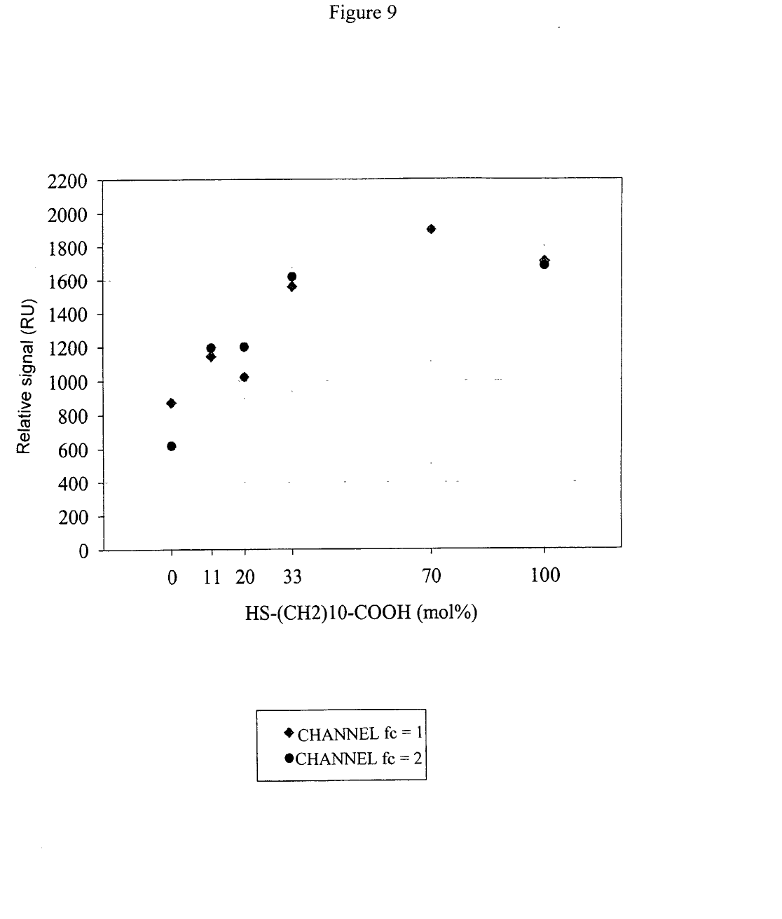

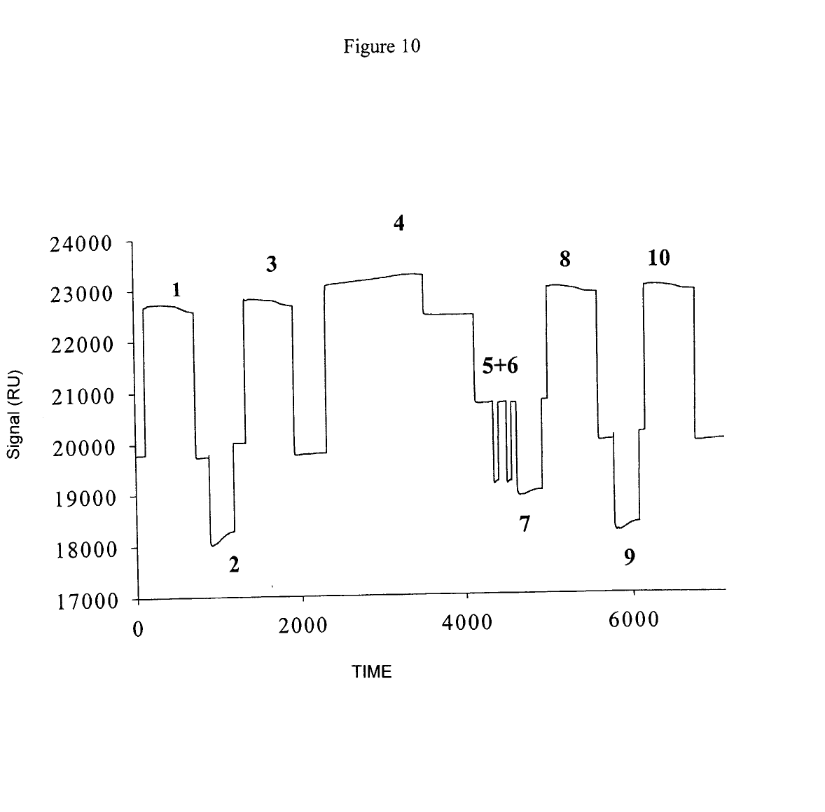

FIG. 9 shows the amount of PEG-PE anchored, i.e the RU signal intensity observed before the inactivation step (step 8) for all surfaces Sia-0, -11, -20, -33, -70, -100%. The coupling reaction is carried out on two different channels (fc=1, fc=2) for a given surface.

For fc=1 and fc=2, the amount of PEG-PE anchored was again found to be similar for all the surfaces. The level of coupling decreased in proportion to the percentage of —COOH functional groups exposed at the surface. The overall decrease is small (maximum≅1800 RU, minimum±1200 RU). The most significant variations were found for the surfaces Sia-33, Sia-20, Sia-11%.

When the experiment is carried out on the surface functionalized only with —OH hydroxyl groups that cannot be activated, the mean level of anchoring of PEG-PE is equal to 718 RU. When the activator EDC/NHS (injection 3) is replaced by a simple injection of HBS-N buffer, the signal is equal to 570 RU. As the —OH groups do not in principle react with the activator, the observed signal may therefore correspond to intercalation of the phospholipid at the SAM and/or baseline stray during the experiment.

### EXAMPLE 4

### Formation of a Bilayer Membrane

Construction of the membrane may be envisioned according to two methods:

that involving dilution of micelles

that involving fusion of vesicles

The vesicle dilution method is addressed in this example.

The surface is regenerated by simple injection of OG detergent.

The lipid POPC, a membrane constituent, is injected as a micelle solution (2 mg/ml of 50 mM OG in HBS-N buffer).

The procedure developed for membrane assembly is fully automated

| | | |
|---|---|---|
| 1. | 50 mM OG detergent in HBS-N buffer | (100 μl; 10 min) |
| 2. | BSA (0.1 mg/ml) | (50 μl; 10 min) |
| 3. | 50 mM OG detergent in HBS-N buffer | (100 μl; 10 min) |
| 4. | Micelles | |
| | 2 mg of lipids/ml from 50 mM OG in | (100 μl; 20 min) |
| | HBS-N | |
| | 1 mg/ml from 25 mM OG in HBS-N | (50 μl; 10 min) |
| 5. | 20 mM NaOH | (10 μl; 1 min) |
| 6. | 20 mM NaOH | (10 μl; 1 min) |
| 7. | BSA (0.1 mg/ml) | (50 μl; 10 min) |
| 8. | 50 mM OG detergent in HBS-N buffer | (100 μl; 10 min) |
| 9. | BSA (0.1 mg/ml) | (50 μl; 10 min) |
| 10. | 50 mM OG detergent in HBS-N buffer | (100 μl; 10 min) |

The dilution step consists in successively injecting a micellar solution of POPC lipid (2 mg/ml of 50 mM OG in HBS-N buffer) followed by a ½ dilution of the solution in HBS-N buffer.

The BSA injection serves as an indicator of bilayer assembly. Indeed, BSA preferentially binds to hydrophobic surfaces (e.g., surface not presenting a membrane), i.e. before the POPC injection or after regeneration of the surface. A signal <100 RU should be observed in the presence of membrane.

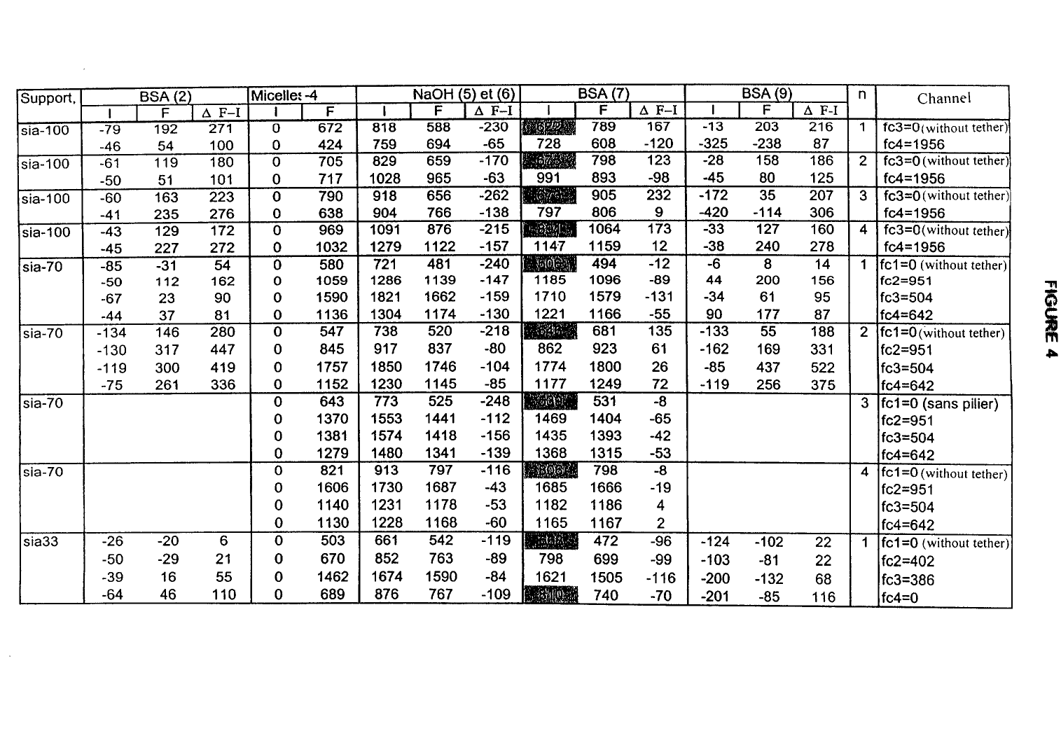

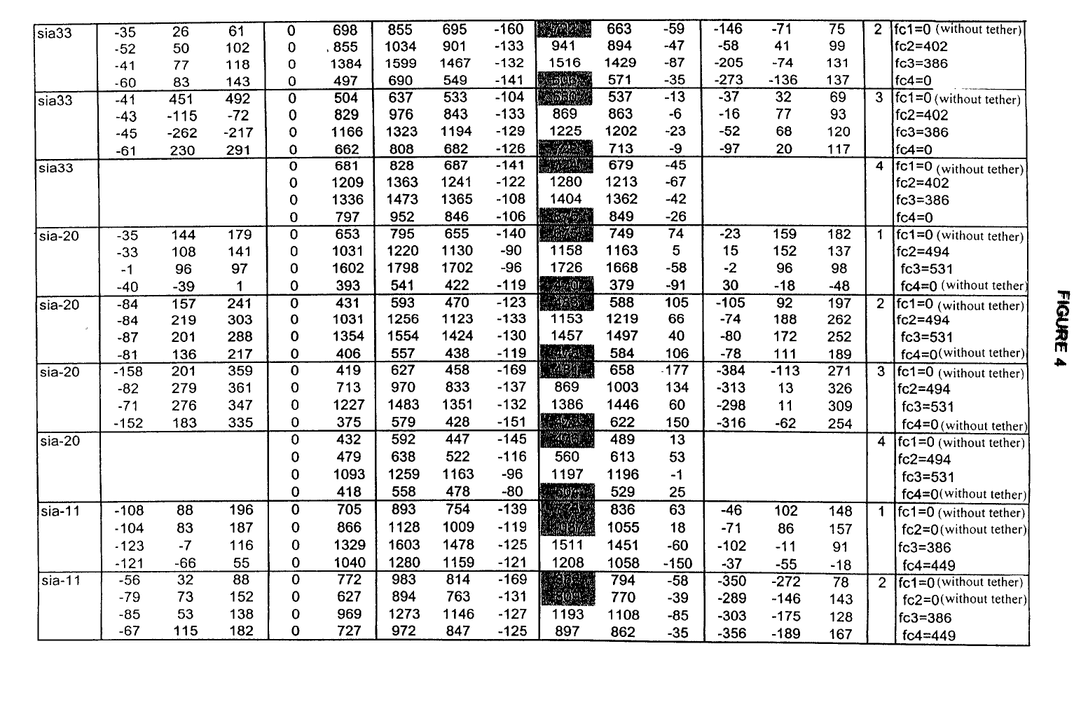

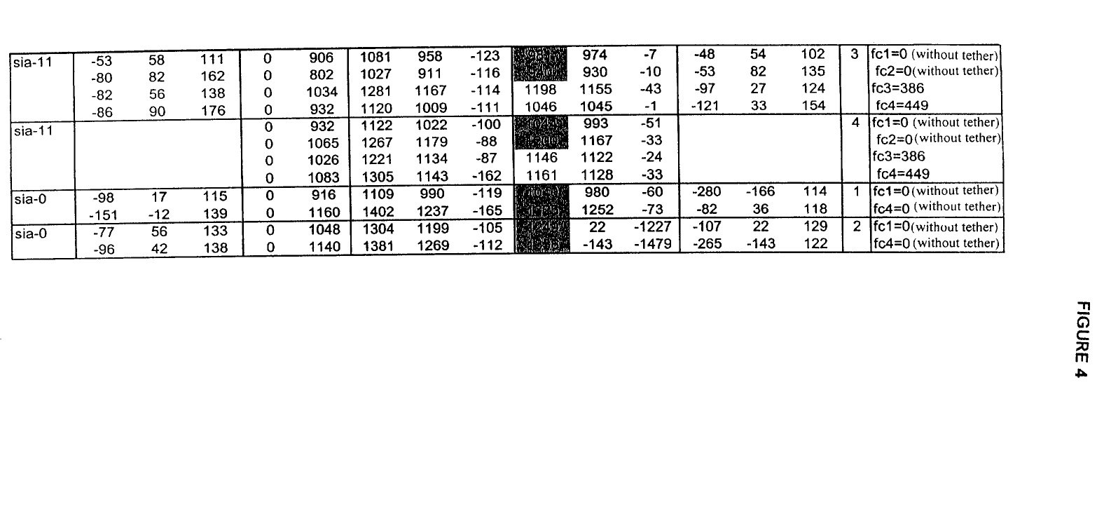

The table in FIG. 4 gives the results obtained for each surface. (I) is the RU signal intensity before injection of a reagent and (F) after injection; the variation (Δ) in the signal corresponds to the difference (F−I). Intensities are reported for injection of BSA before membrane assembly (injection 2), POPC micelles (injection 4), NaOH (injection 5 and 6), BSA after POPC membrane assembly (injection 7) and BSA after regeneration of the surface (injection 9).

The signal corresponding to POPC membrane assembly is obtained after washing the surface with 20 mM NaOH (injection 5 and 6) followed by a return to a stable signal, i.e. column (I) in BSA (7). The intensities of signals corresponding to POPC membrane assembly are given for each surface according to the level of anchored DOPE-C tethers on the different channels. It can be seen that BSA (7) is <100 RU. BSA (2) and (9) are greater than BSA (7).

Sia-100 and -0% : signal intensity after membrane assembly is similar in the presence or absence of the phospholipid tether. The mean signal is 700 and 1200 RU, respectively.

Sia-1 1, -20, -33 and -70%: the increase in the signal after assembly of the POPC membrane is greater in the presence of the DOPE-C (P) tether than in its absence. In the presence of the tether, the increase in the signal corresponding to assembly of the POPC membrane is independent of tether density. The mean variation is from +1000 to 1200 RU.

### EXAMPLE 5

### Test of Anchoring of Membrane Protein

Membrane fragments from CHO cells in which the R-CGRP receptor coupled to the Ramp protein is overexpressed, were applied on the Sia-11% surface. The reaction was conducted in situ in the BIAcore apparatus.

### EXAMPLE 6

### Tether Density

The density of DOPE-C tethers (in RU) on the SAM surface is calculated by considering that a carbon chain has an area of 0.40 nm², that a thiol derivative molecule HS—(CH₂)11-OH or HS—(CH₂)10-COOH has an area of 0.40 nm² and that a DOPE-C phospholipid molecule has an area of 0.80 nm². In this example, a COOH group is also presumed to react with the NH₂ group of the phospholipid to form an amide bond with a yield of 100%.

It is further presumed that the distribution of COOH groups is homogeneous and that the percentage of COOH groups at the SAM surface is identical to that of the solutions used for preparation of the supports.

If the true area occupied by the —COOH group is less than 0.80 nm², the capacity of the SAM is divided in half.

1000 RU is equivalent to 1 ng of phospholipid/mm²

MW of DOPE-C: 879 g/mol

The results are given in the following table.

| | Predicted density | | Found density of | | |
|---|---|---|---|---|---|
| | of DOPE-C tethers | | DOPE-C tethers | | |
| | | (pmol/ | | (pmol/ | Yield |
| Support | (RU) | mm2) | (RU) | mm2) | (%) |
| Sia-100 | 1820 | 2.07 | 1956 | 2.22 | 107 |
| Sia-70 | 1274 | 1.45 | 796 | 0.91 | 62 |
| Sia-33 | 1208 | 1.37 | 394 | 0.45 | 33 |
| Sia-20 | 728 | 0.83 | 512 | 0.58 | 70 |
| Sia-11 | 400 | 0.45 | 417 | 0.47 | 104 |

* mean calculated from two experiments

Considering that the yield of the coupling reaction can range from 70 to 100% but also the approximations in the predicted value, the experimentally observed density of the DOPE-C tethers is close to the predicted value (except for the low value seen with Sia-33)

Stability

The following table shows the variations in RU before and after each injection:

| | | NaOH | NaOH | HCl | |
|---|---|---|---|---|---|
| | POPC | 20 mM | 100 mM | 100 mM | |
| Support | Membrane | 100 μl/min | 10 μl/min | 10 μl/min | Surface |
| sia-100 | 866 | −150 | −55 | −55 | fc1 = 0 |
| | | | | | (without tether) |
| sia-100 | 799 | −92 | −39 | 40 | fc4 = 1956 RU |
| | | | | | (with tether) |
| sia-70 | 961 | −139 | −33 | −28 | fc1 = 0 |
| | | | | | (without tether) |
| sia-70 | 1271 | −129 | −33 | 3 | fc2 = 951 RU |
| | | | | | (with tether) |
| sia-70 | 1641 | −107 | −45 | 71 | fc3 = 504 RU |
| | | | | | (with tether) |
| sia-70 | 1127 | −79 | −43 | 28 | fc4 = 642 RU |
| | | | | | (with tether) |
| sia-33 | 906 | −248 | −46 | −28 | fc1 = 0 |
| | | | | | (without tether) |
| sia-33 | 1263 | −114 | −42 | 3 | fc2 = 402 RU |
| | | | | | (with tether) |
| sia-33 | 1979 | −171 | −57 | 138 | fc3 = 386 RU |
| | | | | | (with tether) |
| sia-33 | 1041 | −107 | −54 | 87 | fc4 = 0 |
| | | | | | (without tether) |
| sia-0 | 1653 | −155 | −48 | −4 | fc1 = 0 |
| | | | | | (without tether) |
| sia-0 | 1157 | −108 | −55 | 125 | fc4 = 0 |
| | | | | | (without tether) |

The stability of the supported membranes formed from POPC lipid are similar for all the surfaces tested under the experimental conditions, i.e: prolonged injection of HBS-N chase buffer, injection of 20 mM NaOH (100 μl/min, 1 min), of NaOH and 100 mM HCl (10 μl/min 10 ul, 1 min).

For a given support, surfaces without tethers are less stable than those with tethers.

1. Method of preparation of an artificial supported membrane comprising the following steps:
a) placing a functionalized surface in the presence of a mixture of specific ligands (L) and phospholipid tethers (P) able to bind to said functionalized surface, and
b) placing the surface formed in a) in the presence of lipids so as to allow the assembly of a lipid bilayer supported by the phospholipid tethers (P).

2. Method according to claim 1, wherein the functionalized surface comprises molecules bearing functional groups allowing covalent binding to the phospholipid tethers (P) and/or specific ligands (L), and molecules bearing non-functional groups.

3. Method according to claim 2, wherein the functionalized surface comprises molecules bearing functional groups binding specifically to the phospholipid tethers (P) and other molecules bearing functional groups binding specifically to the specific ligands (L).

4. Method according to claim 2, wherein the functionalized surface comprises molecules bearing functional groups X, that can possibly be activated, binding to the specific ligands (L) and the phospholipid tethers (P), and molecules bearing non-functional groups Z.

5. Method according to claim 3, wherein the functionalized surface comprises a mixture of molecules bearing functional groups X and Y, the X and Y groups binding selectively to the specific ligands (L) and tethers (P), respectively.

6. Method according to one of claims 4 or 5, wherein the molecules bearing functional or non-functional groups are thiol derivatives represented by the formula HS—(CH₂)n-X, HS—(CH₂)m-Y or HS—(CH₂)n-Z in which n and m, which are the same or different, represent a whole number comprised between 2 and 15.

7. Method of preparation of a supported membrane, comprising the following steps:
a) coating a surface with molecules represented by the formula HS—(CH₂)n-X and HS—(CH₂)m-Z, X being a previously activated functional group capable of coupling with a specific ligand (L) or phospholipid tether (P), Z being a non-functional group incapable of reacting with said ligand (L) or tether (P) and n and m being comprised between 2 and 15,
b) placing the functionalized surface obtained in a) in the presence of a mixture containing specific ligands (L) and phospholipid tethers (P), then
c) placing the surface obtained in b) in the presence of lipids so as to allow the assembly of a lipid bilayer supported by the phospholipid tethers (P).

8. Method of preparation of a supported membrane, comprising the following steps:
a) coating a surface with a mixture of molecules represented by the formula HS—(CH₂)n-X, HS—(CH₂)m-Y and HS—(CH₂)m-Z, X and Y being previously activated functional groups capable of coupling with a phospholipid tether (P) and a specific ligand (L), respectively, Z being a non-functional group incapable of reacting with said ligand (L) or tether (P) and n and m being comprised between 2 and 15,
b) placing the functionalized surface obtained in a) in the presence of a mixture containing specific ligands (L) and phospholipid tethers (P), then
c) placing the surface obtained in b) in the presence of lipids so as to allow the assembly of a lipid bilayer supported by the phospholipid tethers (P).

9. Method according to claim 7 or 8, wherein, in step b), the surface obtained is placed in contact with the phospholipid tethers (P) in a first step, and with the specific ligands (L) in a second step.

10. Method according to one of claims 7 to 9, wherein it comprises an additional step, after step a), to inactivate X or Y groups that did not react with the ligands (L) or tethers (P).

11. Method according to any of the previous claims, wherein it comprises an additional step to insert one or more proteins in the supported membrane.

12. Method according to any of the previous claims, wherein the lipids used in step c) are cell extracts, membrane fragments, micelles, lipid monolayers or bilayers, lipid vesicles and/or lipids, phospholipids, lipopeptides or biotinylated lipids, alone or in mixtures.

13. Method according to any of the previous claims, wherein the surface used is a surface of gold, glass, diamond, silicon, silicon dioxide (SiO₂), silicone nitrite, tantalum pentoxide (Ta₂O₅), titanium dioxide (TiO₂), titanium nitrite, titanium carbide, platinum, tungsten, aluminium or indium-tin oxide, or based on these materials.

14. Method according to claim 13, wherein the surface is a metallic surface, preferably based on or made of gold.

15. Method according to any one of claims 6 to 14, wherein the numbers n and m are equal to 10 or 11.

16. Method according to one of claims 2 to 15, wherein the functional groups X and Y are chosen from among COOH, CHO, OH, NH₂ groups, maleimide and biotin.

17. Method according to one of claims 2 to 16, wherein the molar ratio of the functional groups X and Y to the non-functional groups is comprised between 0.05 and 20, preferably between 0.1 and 5 and even more preferably between 0.2 and 0.3.

18. Method according to any one of the previous claims, wherein the specific ligand (L) comprises a group with specific affinity for a molecule of interest, a point of anchorage to the functionalized surface and a spacer, the length of which is preferably comprised between 3 and 500 Å.

19. Method according to claim 18, wherein the specific ligand (L) comprises a group with specific affinity for a molecule of interest chosen from among a protein, a substrate, an antigen, a hapten, a lectin, a bioreceptor, an oligonucleotide or an immunoglobulin or a region thereof, preferably a protein.

20. Method according to claim 19, wherein the group with specific affinity of ligand (L) is an antibody or antibody fragment specific of a membrane protein.

21. Method according to any one of the previous claims, wherein the phospholipid tether (P) comprises a point of anchorage to the surface, a spacer and a phospholipid.

22. Method according to claim 21, wherein the phospholipid tether (P) comprises a spacer whose length is less than 500 Å, preferably comprised between 3 and 500 Å.

23. Artificial supported membrane wherein it comprises a lipid bilayer attached to a support by phospholipid tethers (P) creating a space between the bilayer and the support, and wherein it comprises one or more ligands (L) specific of a molecule of interest covalently bound to the support and exposed in said space.

24. Artificial supported membrane wherein it may be produced by a method according to one of claims 1 to 22.

25. Use of a supported membrane according to claim 23 or 24, or obtained by a method according to any one of claims 1 to 22, for the purification of membrane proteins.

26. Use of a supported membrane according to claim 23 or 24, or obtained by a method according to any one of claims 1 to 22, for the reversible capture of membrane proteins or membrane protein complexes.

27. Use of a supported membrane according to claim 23 or 24, or obtained by a method according to any one of claims 1 to 22, for the screening of compounds interacting with membrane proteins.

28. Use of a supported membrane according to claim 23 or 24, or obtained by a method according to any one of claims 1 to 22, for the analysis of protein-protein interactions within a membrane.

29. Method of purification of a membrane protein, comprising placing a supported membrane according to claim 23 or 24, or obtained by a method according to any one of claims 1 to 22, in the presence of a membrane preparation comprising the protein to be purified, under conditions allowing introduction of said protein into said supported membrane, and applying a flow of lipids.
